(19) United States
(12) Patent Application Publication
(10) Pub. No.: US 2024/0033538 A1
(43) Pub. Date: Feb. 1, 2024

(54) PHOTOTHERAPY ASSISTANCE METHOD AND PHOTOTHERAPY ASSISTANCE DEVICE

(71) Applicant: OLYMPUS CORPORATION, Tokyo (JP)

(72) Inventor: Ryo MACHIDA, Tokyo (JP)

(73) Assignee: OLYMPUS CORPORATION, Tokyo (JP)

(21) Appl. No.: 18/378,395

(22) Filed: Oct. 10, 2023

Related U.S. Application Data
(63) Continuation of application No. PCT/JP2021/017201, filed on Apr. 30, 2021.

(51) Int. Cl. A61N 5/06

(52) U.S. Cl. CPC A61N 5/062; A61N 2005/0663; A61N 2005/0626

(57) ABSTRACT

A phototherapy assistance method for assisting phototherapy applied to an affected area using a photo-responsive medical agent. The phototherapy assistance method including obtaining blood flow related information regarding blood flow of the affected area, generating progression degree related information regarding progression degree of the phototherapy in the affected area by associating the blood flow related information with the progression degree, and indicating the progression degree related information.

### CROSS-REFERENCE TO RELATED APPLICATIONS

This is a continuation of International Application PCT/JP2021/017201, with an international filing date of Apr. 30, 2021, which is hereby incorporated by reference herein in its entirety.

### TECHNICAL FIELD

The present disclosure relates to a phototherapy assistance method and a phototherapy assistance device.

### BACKGROUND ART

Conventionally, in a known phototherapy, an affected area is treated by irradiating the therapeutic light onto an affected area where the photo-responsive medical agent is administered so as to excite the medical agent (See PTL1, for example). Intensity of fluorescence which is generated by the medical agent decreases as the phototherapy progresses. In PTL1, when the intensity of the fluorescence becomes lower than or equal to a predetermined threshold, the irradiation of the therapeutic light onto the affected area is terminated.

### CITATION LIST

### Patent Literature

{PTL 1} Japanese Unexamined Patent Application, Publication No. 2006-167046

### SUMMARY

An aspect of the present disclosure is a phototherapy assistance method for assisting phototherapy applied to an affected area using a photo-responsive medical agent, the phototherapy assistance method including: obtaining blood flow related information regarding blood flow of the affected area based on reflected light of detection light that is irradiated onto the affected area when therapeutic light is being irradiated onto the affected area, a wavelength of the detection light being different from that of the therapeutic light irradiated onto the affected area to which the a photo-responsive medical agent is applied; generating progression degree related information regarding progression degree of the phototherapy in the affected area by associating the blood flow related information with the progression degree; and indicating the progression degree related information.

Another aspect of the present disclosure is a phototherapy assistance system for assisting phototherapy applied to the affected area using the photo-responsible medical agent, the phototherapy assistance system including: a blood flow related information obtaining section which obtains blood flow related information regarding blood flow of the affected area based on reflected light of detection light that is irradiated onto the affected area when therapeutic light is being irradiated onto the affected area, a wavelength of the detection light being different from that of the therapeutic light irradiated onto the affected area to which the a photo-responsive medical agent is applied; an information generating section which associates the blood flow related information with progression degree of the phototherapy in the affected area to generate progression degree related information regarding the progression degree; and an indication section which indicates the progression degree related information.

### BRIEF DESCRIPTION OF DRAWINGS

FIG. 1 is an overall configuration view of a phototherapy assistance device and a phototherapy system according to a first embodiment of the present disclosure.

FIG. 2A is a flowchart of phototherapy using the phototherapy system of FIG. 1.

FIG. 2B is a flowchart of a phototherapy progression degree determination routine of FIG. 2A.

FIG. 3 is a view showing one example of an endoscope image displayed on a monitor during the phototherapy.

FIG. 4 is an overall configuration view of the phototherapy assistance device and the phototherapy system according to a second embodiment of the present disclosure.

FIG. 5 is a flowchart of a phototherapy progression degree determination routine in a phototherapy method using the phototherapy system of FIG. 4.

FIG. 6 is an overall configuration view of a modified example of the phototherapy system of FIG. 4.

FIG. 7 is an overall configuration view of a modified example of the phototherapy system of FIG. 1.

FIG. 8A is a figure showing one usage example of another modified example of the phototherapy system according to the first and the second embodiments.

FIG. 8B is a figure showing another usage example of another modified example of the phototherapy system according to the first and the second embodiments.

### DESCRIPTION OF EMBODIMENTS

### First Embodiment

A phototherapy assistance device, a phototherapy system, and a phototherapy assistance method according to a first embodiment of the present disclosure will be described below with reference to the accompanying drawings.

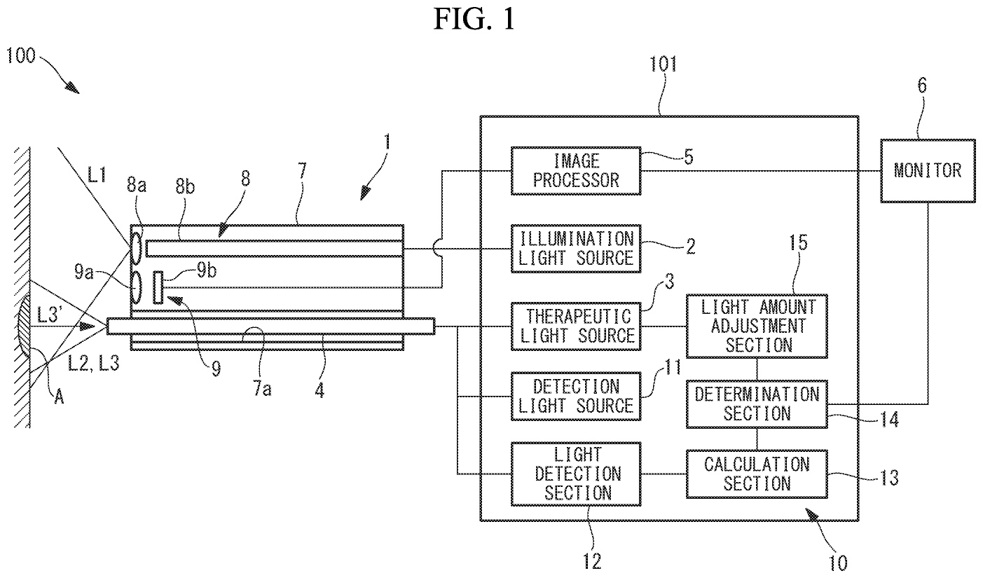

As shown in FIG. 1, a phototherapy system 100 according to this embodiment is an endoscope system that applies phototherapy to an affected area A using a photo responsive medical agent while observing the affected area A by means of an endoscope 1. The affected area A is an upper digestive tract such as an esophagus with cancer, for example. The agent is a fluorescent molecule having a property of being accumulated in the affected area A and is activated by being excited by exciting light to provide a therapeutic effect. The agent is a Pan-IR700 or a hematoporphyrin derivative, for example.

The phototherapy system 100 includes an endoscope 1, an illumination light source 2, a therapeutic light source 3, a probe 4, an image processor 5, and a monitor 6. The light sources 2, 3 and the image processor 5 are provided in an endoscope processor 101 which is connected to a proximal end of the endoscope 1.

The endoscope 1 has a long insertion portion 7 which is flexible or rigid, an illumination optical system 8 and an optical imaging system 9 which are provided in the insertion portion 7. The insertion portion 7 is provided with a treatment instrument channel 7a which penetrates thought the insertion portion 7 in a longitudinal direction.

The illumination optical system 8 guides illumination light L1, which is output from the illumination light source 2, from a proximal end of the insertion portion 7 to its distal end and emits the illumination light L1 to the affected area from the distal end of the insertion portion 7. For example, the illumination optical system 8 includes an illumination lens 8a which is arranged at the distal end of the insertion portion 7 and a light guide 8b which is arranged along almost the whole length of the insertion portion 7, and the illumination light source 2 supplies the illumination light L1 to a proximal end of the light guide 8b.

The optical imaging system 9 takes images of visual field including the affected area A and obtains endoscope images. For example, the optical imaging system 9 has an objective lens 9a arranged at the distal end of the insertion portion 7 and an imaging element 9b arranged at a proximal end side of the objective lens 9a.

The illumination light source 2 generates white light which is a continuous light as the illumination light L1 and outputs the illumination light L1.

The therapeutic light source 3 emits therapeutic light L2 which is a continuous light for exciting the agent to generate fluorescence and outputs the therapeutic light L2. That is, the therapeutic light L2 is exciting light having excitation wave length of the agent, and is red light, for example.

The probe 4 is a long optical fiber probe having an optical fiber for guiding the therapeutic light L2 and is inserted within the treatment instrument channel 7a. The proximal end of the probe 4 is connected to the therapeutic light source 3, and the therapeutic light L2 is irradiated onto the affected area A from the distal end of the probe 4.

The image processor 5 receives the endoscope images from the optical imaging system 9 and outputs the endoscope images to the monitor 6 after processing on the endoscope images if necessary.

The monitor 6 displays the endoscope images input from the image processor 5.

Further, the phototherapy system 100 has a phototherapy assistance device 10 which determines progress of the phototherapy in the affected area A based on blood flow related information regarding the blood flow of the affected area A. The blood flow of the affected area A changes depending on the progress of the phototherapy in the affected area A. In other words, the number of capillary vessels is increased in a surface layer of the affected area A such as an area with cancer. As the phototherapy progresses, the number of the capillary vessels of the affected area A decreases or blood flow through the capillary vessels slows or stops, which changes the blood flow of the affected area A. Accordingly, the blood flow related information is a parameter value indicating the progress of the phototherapy in the affected area A, and it is possible to quantify the progress of the phototherapy by using the blood flow related information.

The phototherapy assistance device 10 of the present embodiment uses a blood flow rate of the affected area A as the blood flow related information. More specifically, the phototherapy assistance device 10 includes a detection light source 11 which generates the detection light L3, a detection light irradiation section 4 which emits the detection light L3 to the affected area A, a light detection section 12 which detects detection light L3′ which is reflected by the affected area A, a calculation section 13 which calculates the blood flow rate of the affected area A based on the amount of the detection light L3′, a determination section (an information generating section) 14 which compares the blood flow rate with a predetermined threshold Th and generates support information, a indication section 6 which provides the support information, and a light amount adjusting section 15 which adjusts the amount of the therapeutic light irradiated onto the affected area A based on a detected result of the determination section 14.

The detection light source 11, the light detection section 12, the calculation section 13, the determination section 14, and the light amount adjustment section 15 are provided in an endoscope processor 101. The phototherapy assistance device 10 includes at least one processor and a memory provided in the endoscope processor 101. The processor executes the later described processing according to a phototherapeutic support program stored in the memory so that the later described features of the calculation section 13, the determination section 14, and the light amount adjusting section 15 are realized.

The detection light source 11, the detection light irradiation section 4, the light detection section 12, and the calculation section 13 composes a blood-flow related information obtaining section which obtains blood flow rate of the affected area A as a parameter for determination used for determination conducted by the determination section 14.

The detection light source 11 generates the detection light L3 for detecting the blood flow rate of the affected area A, and outputs the detection light L3. The detection light L3 is a continuous light absorbed by blood. Preferably, the detection light L3 is a light including blue light, and the detection light L3 is a blue narrow band light, for example. The blue light has a feature to be reflected strongly by the surface layer of the affected area A and to be absorbed strongly in the blood of the capillary vessel in the surface layer of the affected area A. Accordingly, the amount of the blue light included in the detection light L3′ reflected by the affected area A correlates with the blood flow rate in the capillary vessel in the surface layer of the affected area A.

The detection light irradiation section 4 is the above described probe, and also the proximal end of the probe 4 is connected to the detection light source 11. The detection light L3 is guided by the probe 4 which is the same probe for guiding the therapeutic light L2, and the detection light L3 is irradiated onto the affected area A from the distal end of the probe 4. The proximal end of the probe 4 is connected to the light detection section 12, the detection light L3′ reflected by the affected area A is received by the distal end of the probe 4 and is guided to the light detection section 12. The therapeutic light L2, the detection light L3, and detection light L3′ may be guided by a mutual optical fiber or they may be guided by different optical fibers.

The light detection section 12 has a light detection device (not shown) of any kinds. The light detection section 12 receives the detection light L3′ which enters into the light detection section 12 from the probe 4, the amount of the detection light L3′ (more specifically, it is the amount of the blue light included in the detection light L3′) is detected by the light detection device, and information of the amount of the light is sent to the calculation section 13.

The calculation section 13 calculates the blood flow rate of the affected area A based on the amount of the detection light L3′. The greater the blood flow rate of the affected area A becomes, the more the affected area A absorbs the detection light L3 and therefore the lower the amount of the detection light L3′ received by the distal end of the probe 4 becomes. From this, the calculation section 13 is capable of calculating the blood flow rate of the affected area A based on the amount of the detection light L3. For example, the calculation section 13 has a table or a formula indicating a correspondence relationship between the amount of the detection light L3 and the blood flow rate, and uses the table or the formula to convert the amount of the detection light L3 into the blood flow rate.

The calculation section 13 may calculate the absorbance of the affected area A with respect to the detection light L3 (absorbance per unit area of the affected area A, for example) instead of the blood flow rate. The absorbance of the affected area A correlates with the blood flow rate. Therefore, it is possible to use the absorbance in the later described determination by the determination section 14 instead of the blood flow rate.

The determination section 14 compares the blood flow rate and the predetermined threshold Th to determine whether or not the blood flow rate reaches the threshold Th. The predetermined threshold Th is a value of 10% of the blood flow rate at the start of the phototherapy, for example. It is preferable that this threshold Th is set to be a desired value for each affected area A by a doctor grasping a state of the affected area A beforehand. A plurality kinds of thresholds may be provided. Moreover, the blood flow related information obtaining section may obtain the blood flow rate of the affected area A at the start of the phototherapy and compare the obtained blood flow rate and a targeted blood flow rate, and may set the threshold Th so that the ratio of the blood flow rate becomes a desired ratio.

The determination section 14 generates the support information depending on the detection result and sends the support information to the monitor 6. The support information obtained by comparing the blood flow rate with the threshold Th is progression degree related information regarding the progress of the phototherapy to the affected area A. For example, when the blood flow rate is determined to be greater than the threshold Th, the support information can be an instruction indication for continuing the phototherapy. When the blood flow rate is determined to be lower than the threshold Th, the support information could be an indication asking whether the phototherapy should be terminated or not. Also, the determination section 14 outputs the information on the determination result to the light amount adjustment section 15.

The indication section 6 is the above mentioned monitor, which notifies the doctor the detection result as to whether the blood flow rate reaches the set threshold Th by displaying the support information received from the determination section 14. The doctor can determine whether or not the phototherapy has completed by considering the determination result by the determination section 14.

When the determination section 14 determines that the blood flow rate is lower than the threshold Th, the light amount adjustment section 15 controls the therapeutic light source 3 to reduce the output of the therapeutic light L2 from the therapeutic light source 3 or stop the output of the therapeutic light L2 in order to reduce the amount of therapeutic light L2 irradiated onto the affected area A. Here, the threshold used for determination by the doctor and the threshold for reducing the amount of the therapeutic light L2 from the therapeutic light source 3 may be different. For example, it is preferable to avoid excessive radiation of the light to the affected area A by setting lower threshold for reducing the amount of therapeutic light L2 from the therapeutic light source 3 than the threshold used for determination by the doctor.

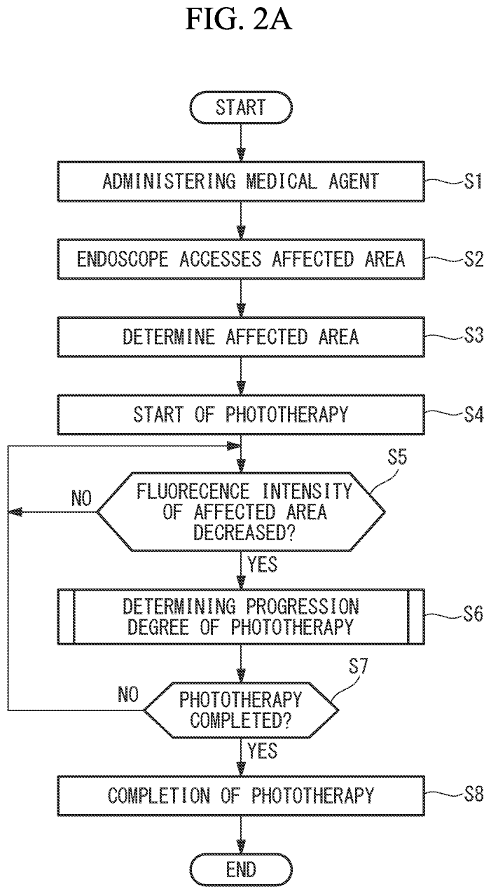

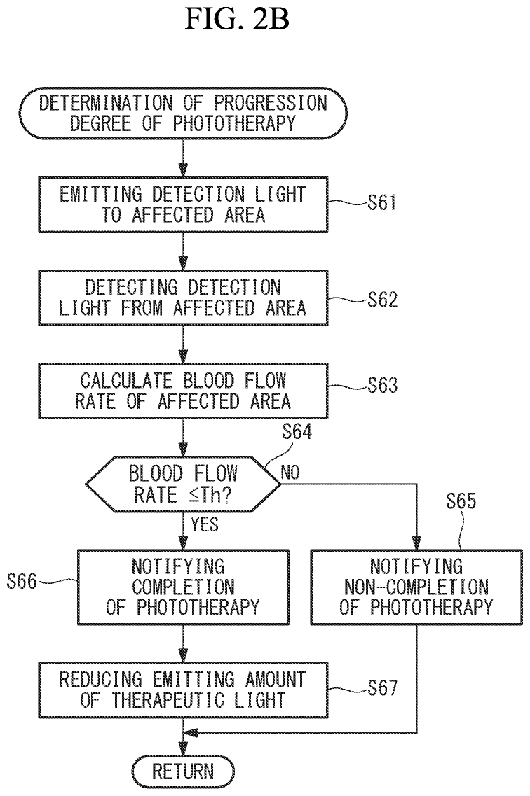

Subsequently, a phototherapy method using the phototherapy system 100 will be described below with reference to FIGS. 2A and 2B.

As shown in FIG. 2A, the phototherapy method according to the present embodiment includes Step S1 for administering the agent to the affected area A, Step S2 for accessing the affected area A with the endoscope 1, Step S3 for determining the affected area A based on the endoscope images, and Steps S4 to S8 for performing phototherapy to the affected area A.

In Step S1, an operator such as a doctor, a nurse, or the like administers the agent to the affected area A by giving an intravenous injection to a patient, for example. After the intravenous injection is given, it takes some time for the agent to accumulate in the affected area A. For that reason, the next Step S2 will be conducted after a predetermined time (24 hours, for example) from the intravenous injection.

The agent may be administered in any other ways, and the agent may be administered by directly injecting it into the affected area A, for example. It may be possible to change the timing of Step 1 depending on the administration method. For example, after determining the affected area A in Step 3, the agent may be injected directly to the determined affected area A.

Then, in Step S2, the doctor turns on the illumination light source 2, inserts the endoscope 1 into the body of the patient by observing the endoscope image displayed on the monitor 6, and places the distal end of the endoscope 1 at a position which is close to the affected area A.

Subsequently, in Step S3, the doctor observes the endoscope image displayed on the monitor 6 and determines the affected area A to give the phototherapy. When necessary, the doctor may irradiate the therapeutic light L2 onto a tissue in the body and determine the affected area based on fluorescence.

Then, in Step 4, the doctor inserts the probe 4 into the body through the treatment instrument channel 7a of the endoscope 1, places the distal end of the probe 4 at a position close to the affected area, and turns on the therapeutic light source 3. By this, the distal end of the probe 4 starts to irradiate the therapeutic light L2 onto the affected area A, and the phototherapy is started to be applied to the affected area A.

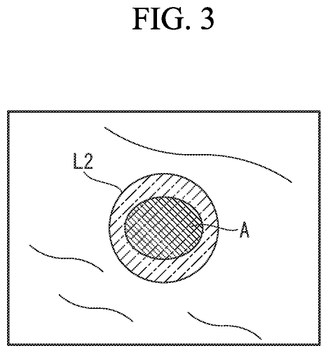

FIG. 3 shows an example of the endoscope image displayed on the monitor 6 during the phototherapy of the affected area A. The affected area A generates fluorescence by irradiation of the therapeutic light L2, and intensity of the fluorescence is reduced as the phototherapy of the affected area progresses. During the phototherapy to the affected area A, the doctor observes the fluorescence of the affected area in the endoscope image displayed on the monitor 6 so that the doctor can confirm that the phototherapy has progressed based on the attenuation of the intensity of the fluorescence. However, it is difficult to determine whether the phototherapy is completed or not based on the intensity of the fluorescence since the intensity of the attenuated fluorescence is small and unstable.

Therefore, after the intensity of the fluorescence is sufficiently reduced when compared with that at the start of the phototherapy (YES in Step S5), the doctor makes the phototherapy assistance device 10 execute Step S6 to confirm the progress of the phototherapy to the affected area. Step S6 is a phototherapeutic method of the present invention for determining the progress of the phototherapy, which is started when the doctor turns on the detection light source 11, for example.

As shown in FIG. 2B, Step 6 includes Steps S61 to S63 for obtaining blood flow rate of the affected area A, Step 64 for determining whether or not the blood flow rate reaches the predetermined threshold Th, Steps S65, S66 for notifying the doctor of the progress of the phototherapy by creating and providing the support information based on the detected result, and Step S67 for adjusting the amount of the therapeutic light L2 with which the affected area A is irradiated.

The step for obtaining the blood flow rate of the affected area A includes Step S61 for irradiating the affected area A with the detection light L3, Step S62 for detecting the detection light L3′ reflected by the affected area A, and Step S63 for calculating the blood flow rate of the affected area A based on the detected detection light L3′.

The detection light L3 output from the detection light source 11 is irradiated onto the affected area A via the probe 4 (Step S61). The detection light L3′ reflected by the affected area A is received by the distal end of the probe 4 and guided to the light detection section 12 by the probe 4, then the amount of the detection light L3′ is detected by the light detection section 12 (Step S62). Then, the calculation section 13 calculates the blood flow rate of the affected area A based on the amount of the detection light L3′.

Subsequently, the determination section 14 compares the blood flow rate with the predetermined threshold Th (Step S64).

When it is determined that the blood flow rate is greater than the predetermined threshold Th (NO in Step S64), the determination section 14 creates the support information for instructing continuation of the phototherapy and displays the support information on the monitor 6 (Step S65). By this, the doctor is notified that the phototherapy has not been completed.

Whereas, when it is determined that the blood flow rate is below the predetermined threshold Th (YES in Step S64), the determination section 14 creates the support information which asks whether to terminate the phototherapy and displays the support information on the monitor 6 (Step S66). By this, the doctor is notified that the phototherapy to the affected area A has been completed. Also, the amount of the therapeutic light L2 irradiated onto the affected area A is reduced by the light amount adjustment section 15 (Step S67).

Then, in Step S7, the doctor determines whether the phototherapy should be terminated or not based on the support information displayed on the monitor 6. More specifically, when the support information that instructs continuation of the phototherapy is displayed, the doctor continues the radiation of the therapeutic light L2 to the affected area A, and after that, the doctor makes the phototherapy assistance device 10 execute the Step 5 again. However, when the support information that asks whether the phototherapy should be terminated or not is displayed, the doctor terminates the irradiation of the therapeutic light L2 onto the affected area A by turning off the therapeutic light source 3 and complete the phototherapy to the affected area A in Step S8.

As explained above, the doctor can recognize that the phototherapy in the affected area A is progressing based on the decrease in the intensity of the fluorescence of the affected area A during the phototherapy, however, it is difficult to judge accurately whether or not the phototherapy in the affected area A using the medical agent is completed based only on the intensity of the fluorescence.

According to this embodiment, the blood flow related information obtaining section obtains the blood flow rate of the affected area A. The blood flow rate of the affected area A is highly correlated with the progression of the phototherapy. Accordingly, it is possible to quantify the current progression of the phototherapy in the affected area A using the blood flow rate of the affected area A, and it is possible to accurately determine whether or not the phototherapy in the affected area A is completed based on the blood flow rate of the affected area A.

Also, the highly reliable support information which is related to the progression of the phototherapy in the affected area A is generated based on the determination result as to whether or not the blood flow rate of the affected area A reaches the predetermined threshold Th, and the support information is provided to the doctor. The doctor can adequately determine when to terminate the phototherapy based on the highly reliable support information and irradiate the therapeutic light L2 onto the affected area A without excess or deficiency. For example, when the support information for instructing continuation of the phototherapy is displayed, the doctor keeps irradiating the therapeutic light L2 onto the affected area A according to the support information, and when the support information asking whether the phototherapy should be terminated or not is displayed, the doctor can terminate the phototherapy applied to the affected area A immediately.

Also, once the phototherapy in the affected area A is completed, the doctor can start to apply the phototherapy to another affected area A immediately, which reduces overall time for the therapy.

Also, according to the present embodiment, the detection light L3 is irradiated onto the affected area A after the intensity of the fluorescence of the affected area A is attenuated. This can prevent unnecessary irradiation of the detection light L3 onto the affected area A.

Moreover, according to the present embodiment, when it is determined that the blood flow rate of the affected area A is lower than or equal to the predetermined threshold Th, the amount of therapeutic light L2 irradiated onto the affected area A is reduced automatically. By this, it is possible to further reduce excessive irradiation of the therapeutic light L2 onto the affected area A.

Also, according to the present embodiment, the detection light L3 has a wavelength different from the therapeutic light L2 and the fluorescence, and therefore, in Step S61, the detection light L3 is irradiated onto the affected area A in a state where the detection light L3 is not interfered by the therapeutic light L2. Further, the detection light L3 is irradiated onto the affected area A after the intensity of the fluorescence is sufficiently reduced. Accordingly, even when the detection light L3 is irradiated onto the affected area A where the therapeutic light L2 is being irradiated, the detection light L3′ can be detected separately from the therapeutic light L2 and the fluorescence.

In Step S61, the detection light L3 may be irradiated onto the affected area A in a state where the therapeutic light L2 is not irradiated onto the affected area A or a state where the reduced therapeutic light L2 is irradiated onto the affected area A by temporality stopping or reducing the therapeutic light L2. This will further prevent the detection light L3 from being interfered by the therapeutic light L2.

In the present embodiment, the detection light L3 may further include light whose wavelength is longer than the blue light. Preferably, the long wavelength light is green light, and for example, it is green narrow band light. The green light has such characteristics that is reflected strongly by a deep position located deeper than the surface layer of the affected area A, and that is strongly absorbed by blood in the blood vessel located at the deep position of the affected area A. Accordingly, it is possible to calculate the blood flow rate at the deep position of the affected area A from the amount of the green light included in the detection light L3′. Furthermore, it is possible to determine whether or not the phototherapy in the affected area A is completed more adequately based on the blood flow rates of the surface layer and the deep position.

In the present embodiment, the blood flow related information obtaining section obtains the blood flow rate based on the amount of the detection light L3′ reflected by the affected area A, however, instead of this, the blood flow rate may be obtained by using a laser Doppler method.

That is, the detection light source 11 outputs a pulsed laser beam as the detection light L3, and the pulsed laser beam is in a wavelength range which is less absorbable by the blood and easily reflected by the blood (in a near infrared region, for example), and the light detection section 12 detects the detection light L3′ reflected by the affected area A via the probe 4. A frequency of the detection light L3′ reflected by the blood is shifted by a shifted amount Δf depending on speed of the blood flow according to the Doppler shift. The calculation section 13 calculates the shifted amount Δf and calculates the blood flow rate of the affected area A from the shifted amount Δf.

In this case, the detection light L3 which is pulsed light can be detected separately from the therapeutic light L2 which is the continuous light. Therefore, the detection light L3 may be irradiated onto the affected area A where the therapeutic light L2 is being irradiated.

### Second Embodiment

Subsequently, a phototherapy assistance device, a phototherapy system, and a phototherapy assistance method according to a second embodiment of the present invention will be described below with reference to the accompanying drawings.

The phototherapy system 200 according to the present embodiment is different from the phototherapy system 100 of the first embodiment in that a phototherapy assistance device 20 obtains a number of capillary vessels of the affected area A as the blood flow related information based on the endoscope image. This embodiment describes differences from the first embodiment, and the same components as those in the first embodiment are accompanied by the same reference numerals and their explanation will be omitted.

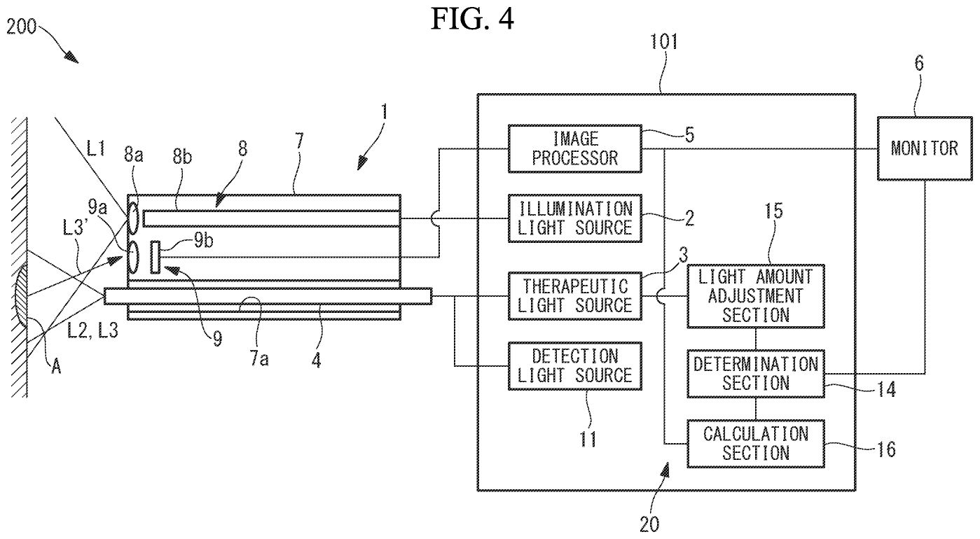

As shown in FIG. 4, the phototherapy system 200 includes an endoscope 1, an illumination light source 2, a therapeutic light source 3, a probe 4, an image processor 5, a monitor 6, and a phototherapy assistance device 20.

The phototherapy assistance device 20 has a detection light source 11, a detection light illumination section 4, a light detection section 9, a calculation section 16, a determination section 14, an indication section 6, and a light amount adjustment section 15.

The light detection section 9 is an optical imaging system (an image obtaining section) of the endoscope 1. When the detection light L3, which is the blue light, is irradiated onto the affected area A, the optical imaging system 9 detects the detection light L3′ which is reflected by the affected area A and obtains a NBI image (image information) which is the endoscope image of the affected area A according to the reflected light L3′. The detection light L3 may further include green light. In the NBI image, a color of the capillary vessels in the surface layer of the affected area A is brown and a color of vessels in the deep part of the affected area A is blue.

The calculation section 16 receives the NBI image from the optical imagining system 9 via the image processor 5, extracts the capillary vessels in the surface layer based on the colors in the NBI image, and calculates the numbers of the extracted capillary vessels in the surface layer. The number of the capillary vessels is a parameter for determination used for determination by the determination section 14. For example, the calculation section 16 generates an image of the capillary vessels by extracting the capillary vessels in the surface layer from the NBI image, binaries the image of the capillary vessels, and calculates an area ratio of the capillary vessels in the image of the capillary vessels as the number of the capillary vessels. When the detection light L3 includes the green light, the calculation section 16 may extract blood vessels located in the deep part based on the colors in the NBI image to calculate the number of the blood vessels of the deep part.

The determination section 14 compares the number of the capillary vessels calculated by the calculation section 16 with a predetermined first threshold Th1 and a predetermined second threshold Th2 to determine whether or not the number of the capillary vessels is lower than or equal to the predetermined first threshold Th1 and greater than or equal to the predetermined second threshold Th2. The first threshold Th1 is equivalent to a value indicating completion of the phototherapy in the affected area A. For example, the first threshold Th1 is the same value as the predetermined threshold Th of the first embodiment, and is a value which is 10% of the number of the capillary vessels at the start of irradiation of the therapeutic light L2. The second threshold Th2 is a value smaller than the first threshold Th1 and is equivalent to a value indicating limit of the phototherapy in the affected area A.

The determination section 14 generates the support information corresponding to the determination result and sends the support information to the monitor 6. The support information obtained by comparing the blood flow rate with the thresholds Th1 and Th2 is progression degree related information regarding the progression of the phototherapy in the affected area A. For example, support information at the time when it is determined that the number of the capillary vessels is smaller than the first threshold Th1 can be an indication instructing continuation of the phototherapy.

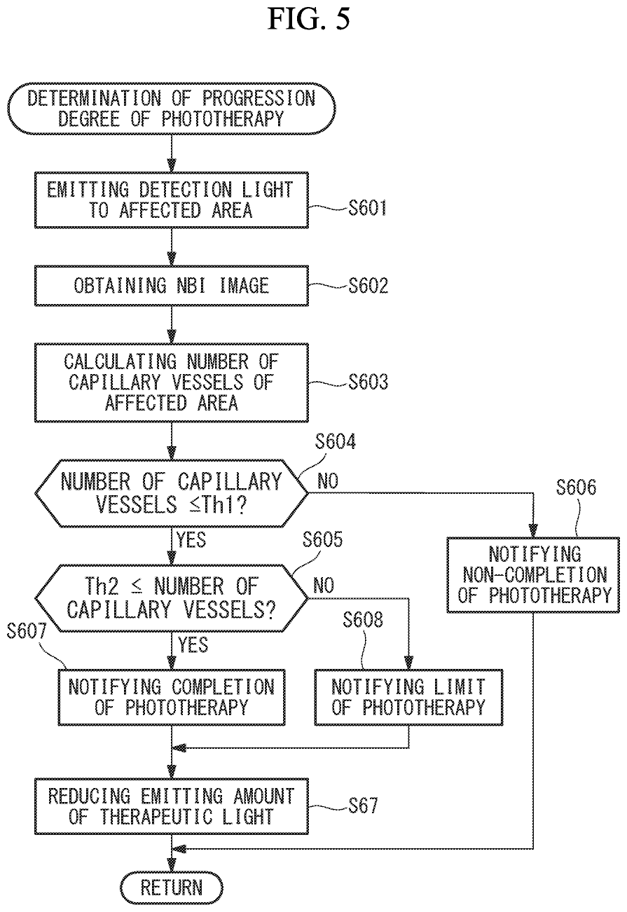

Then, a phototherapy method using the phototherapy system 200 will be described below with reference to FIG. 5.

The phototherapy method according to the present embodiment includes Steps S1 to S8, which are the same as those the first embodiment, however, what is performed in Step S6 is different from that in the first embodiment.

As shown in FIG. 5, Step S6 includes Steps S601 to S603 which obtain the number of the capillary vessels in the affected area A, Steps S604 and S605 which determine whether or not the number of the capillary vessels is lower than or equal to the first threshold Th1 and greater than or equal to the second threshold Th2, Steps S606 to 608 which inform a doctor of the progression of the phototherapy by creating and indicating the support information based on the detected result, and step S67.

The step which obtains the number of the capillary vessels in the affected area A includes Step S601 which irradiates the detection light L3 onto the affected area A, Step S602 which detects the detection light L3′ reflected by the affected area A and obtains the NBI image based on the detection light L3′, and Step S603 which extracts the number of the capillary vessels in the surface layer of the affected area A from the NBI image.

The detection light L3 which is output from the detection light source 11 is irradiated onto the affected area A via the probe 4 (Step S601). As in Step 61 of the first embodiment, in a state where the therapeutic light L2 is not irradiated onto the affected area A or a state where the reduced therapeutic light L2 is irradiated onto the affected area A, the detection light L3 may be irradiated onto the affected area in Step SS601. The detection light L3′ reflected by the affected area A is detected by the optical imaging system 9 of the endoscope 1, and the NBI image is captured by the optical imaging system 9 (Step S602). Then, the calculation section 13 calculates the number of the capillary vessels in the affected area A from the NBI image (Step S603).

Subsequently, the determination section 14 compares the number of the capillary vessels with the thresholds Th1 and Th2 (Steps S604 and 605).

When it is determined that the number of the capillary vessels is greater than or equal to the first threshold Th1 (NO in Step S604), the support information instructing continuation of the phototherapy is generated by the determination section 14, and the support information is displayed on the monitor 6 (Step S606). By this, the doctor is notified that the phototherapy has not been completed.

When it is determined that the number of the capillary vessels is smaller than or equal to the first threshold Th1 and greater than or equal to the second threshold Th2 (YES in Step S604 and YES in Step S605), the support information asking whether the phototherapy should be terminated is generated by the determination section 14, and the support information is displayed on the monitor 6 (Step S607). By this, the doctor is notified that the phototherapy on the affected area has been completed.

When it is determined that the number of the capillary vessels is lower than the second threshold Th2 (YES in Step S604 and NO in Step S605), the information support information instructing termination of the phototherapy is generated by the determination section 14, and the support information is displayed on the monitor 6 (Step S608). By this, the doctor is notified that it reaches the limit of the phototherapy of the affected area A.

Then, in Step S7, the doctor determines whether or not the phototherapy should be terminated based on the highly reliable support information displayed on the monitor 6. More specifically, when the support information instructing continuation of the phototherapy is displayed, the doctor keeps irradiating the therapeutic light L2 onto the affected area A, and after that, the doctor operates the phototherapy assistance device 20 to execute the Step S5 again. Whereas, when the support information asking whether the phototherapy should be terminated or not or the support information instructing termination of the phototherapy is displayed, the doctor turns off the therapeutic light source 3 and finishes the therapeutic light L2 emission to the affected area A and terminates the phototherapy applied to the affected area A.

As explained above, according to the present embodiment, the number of the capillary vessels of the affected area A is obtained by the blood flow related information obtaining section. The number of the capillary vessels of the affected area A is highly correlated with the progression of the phototherapy in the affected area A. Accordingly, it is possible to quantify the current progression of the phototherapy in the affected area A using the number of the capillary vessels of the affected area A, and it is possible to accurately determine whether or not the phototherapy in the affected area A is completed based on the number of the capillary vessels. Furthermore, it is possible to generate the highly reliable support information corresponding to the progression of the phototherapy in the affected area A based on the determination result as to whether or not the number of the capillary vessels is lower than or equal to the threshold Th1 and greater than or equal to the threshold Th2.

Moreover, according to the present embodiment, it is possible to determine the progression of the phototherapy in the affected area A more specifically by comparing the number of the capillary vessels with the two thresholds Th1 and Th2. That is, when the number of the capillary vessels is lower than or equal to the first threshold Th1, it is possible to determine that the phototherapy has been completed and that it is a timing to terminate the phototherapy. Also, when the number of the capillary vessels is smaller than the second threshold Th2, it is possible to determine that any further effect of the phototherapy cannot be expected by irradiating the therapeutic light L2 onto the affected area A anymore and it has reached a limit of continuing the phototherapy.

Since the other operations and the effects are the same as those of the first embodiment, their descriptions will be omitted.

In the present embodiment, the determination section 14 compares the number of the capillary vessels with the two thresholds Th1 and Th2, however, instead of this, the number of the capillary vessels may be compared with a single threshold Th which is the same as the first embodiment to determine whether the number of the capillary vessels is lower than or equal to the threshold Th.

Also, in the first embodiment, the determination section 14 may compare the blood flow rate with the two thresholds Th1 and Th2 to determine whether or not the blood flow rate is lower than or equal to the first threshold Th1 and greater than or equal to the second threshold Th2.

In this embodiment, the blood flow related information is the number of the capillary vessels in the affected area A, however, instead of this, it may be a color of the affected area A.

As the phototherapy of the affected area A progresses, the color of the blood flow rate of the affected area A decreases, and because of this, the color of the affected area A changes. For example, as the phototherapy progresses, the affected area A becomes whitish or black red color. For that reason, it is possible to use the color of the affected area A as the blood flow related information regarding the blood flow.

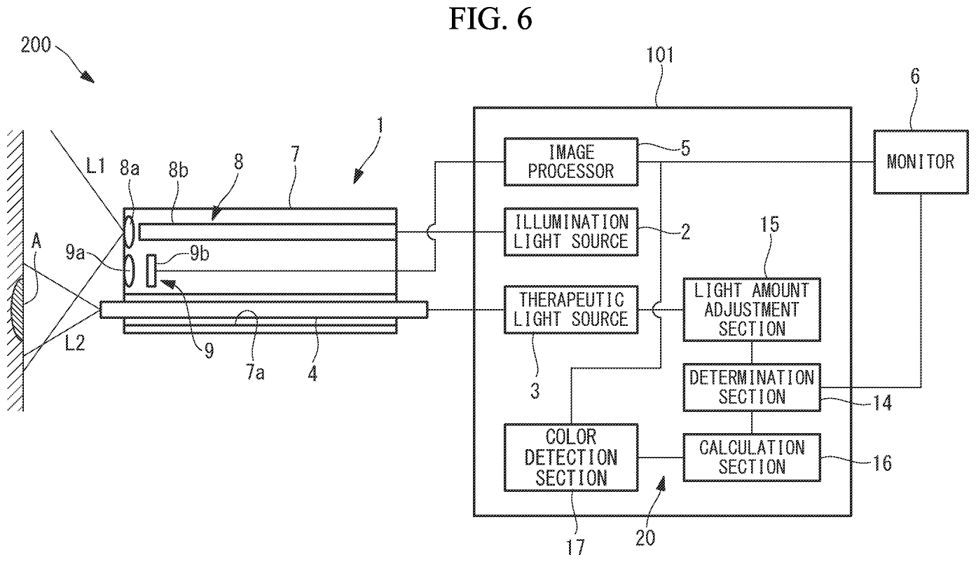

In this case, as shown in FIG. 6, as the blood flow related information obtaining section, the phototherapy assistance device 20 includes the optical imaging system (image obtaining section) 9, a color detection section 17, and the calculation section 16. The optical imaging system 9 obtains a normal captured image (a white light image) which is an endoscope image at the time when the illumination light L1 is irradiated onto the affected area A. The color detection section 17 receives the normal captured image from the optical imaging system 9 via the image processor 5 and detects color of the affected area A which is an area to pay attention to from the normal captured image. For example, the color detection section 17 obtains values of a plurality of color signals constituting each pixel value of the area to pay attention to. When the normal captured image is a RGB image, the plurality of the color signals includes an R signal, a G signal, and a B signal. The area to pay attention to is set based on the fluorescence, and for example, at the time of starting the phototherapy, only the therapeutic light L2 is irradiated onto the affected area A to obtain a fluorescent image as the endoscope image, and the fluorescent area in the fluorescent image is set to be the area to pay attention to.

The blood flow related information obtaining section obtains the color of the affected area A at the start of the phototherapy in Step S4 and stores it in the memory. Then, in Step S6, the optical imaging system 9 obtains the normal captured image, the color detection section 17 detects the color of the affected area A from the normal captured image, and the calculation section 16 calculates a change amount of the color of the affected area A by comparing the detected color with the color of the affected area A at the start of the phototherapy. For example, the change amount of color is a decreased amount of a red component of the affected area A, and the calculation section 16 calculates the change amounts of the R signal, the G signal, and the B signal and calculates a relative decreased amount of the R signal by comparing the change amount of the R signal with the change amounts of the G signal and the B signal.

The determination section 14 compares the change amount of the color with a predetermined threshold, determines whether the change amount of the color is greater than or equal to the predetermined threshold or not, and generates the support information according to the determination result. For example, when the change amount of the color is smaller than the threshold, the determination section 14 generates the support information instructing continuation of the phototherapy, and when the change amount of the color is greater or equal to than the predetermined threshold, the determination section 14 generates the support information asking whether the phototherapy should be terminated or not.

In the first and the second embodiments, the detection light L3 may be pulsed light. With this composition, the detection light L3 can be irradiated onto the affected area A in a state where the detection light L3 is not interfered by the therapeutic light L2. That is, it is possible to detect and distinguish the detection light L3′ separately from the therapeutic light which is the continuous light and the fluorescence when the therapeutic light L2 and the detection light L3 having the same wave length are irradiated onto the affected area A at the same time.

In the first and the second embodiments, the doctor determines the timing of Step S6 according to the reduction of the intensity of the fluorescence of the affected area A, however, it is not limited hereto, and the timing of Step S6 may be changeable in a suitable manner.

For example, Step S6 may be executed automatically at a predetermined time interval according to a predetermined schedule. Alternatively, Step S6 may continuingly be executed from the start to the end of the phototherapy by alternately irradiating the therapeutic light L2 and the detection light L3 onto the affected area A after starting the phototherapy.

Alternatively, the phototherapy assistance devices 10, 20 may execute Step S6 automatically according to the intensity of the fluorescence of the affected area A. For example, the intensity of the fluorescence of the affected area A is measured according to the endoscope image during the phototherapy. When the intensity of the fluorescence is sufficiently reduced, the phototherapy assistance devices 10, 20 automatically execute Step S6. This prevents the detection light L3 from being irradiated unnecessary onto the affected area A before the intensity of the fluorescence is sufficiently reduced.

In the first and the second embodiments, the therapeutic light L2 and the detection light L3 are irradiated onto the affected area A via the mutual probe 4 inserted into the treatment instrument channel 7a, however, the therapeutic light L2 and the detection light L3 may be irradiated onto the affected area A via a different route.

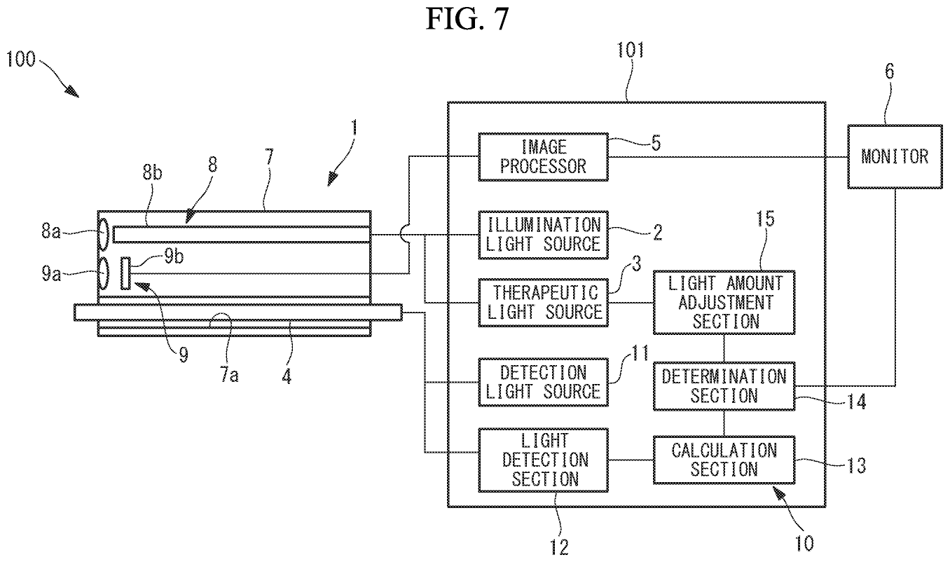

For example, as shown in FIG. 7, the therapeutic light L2 may be irradiated onto the affected area A via the same illumination optical system 8 that is used to generate the illumination light L1.

In the first and the second embodiments, the phototherapy assistance devices 10, 20 are a part of the endoscope system, however, instead of this, the phototherapy assistance devices 10, 20 may be devices which are independent from the endoscope system.

That is, the phototherapy assistance devices 10, 20 may include a detection light irradiation section separate from the probe 4, a light detection section or an image obtaining section separate from the optical imaging system, and a indication section separate from the monitor 6. The indication section may indicate the determination results by other means than displaying the support information, for example, the determination results may be indicated by sound.

In this case, the detection light L3 may be irradiated onto the affected area A via the probe arranged at a position located outside the endoscope 1 and the detection light L3′ is detected by the light detection section arranged at a position located outside the endoscope 1, for example.

In the first and the second embodiments, the blood flow related information obtaining section may further obtain at least one of oxygen saturation around the affected area A, the area, the volume, and the temperature of the affected area A. The obtained information is displayed to the monitor 6. This assists the doctor to apply the phototherapy to the affected area A.

The oxygen saturation around the affected area A is obtained by calculating a spectral characteristic of the affected area A from the image information obtained by the endoscope 1 to detect absorbance difference between oxygenated hemoglobin and reduced hemoglobin.

The area and the volume of the affected area A are obtained by extracting an area having luminescence greater than or equal to a threshold from the fluorescence image as the affected area A and measuring the area and the volume of the affected area A. Alternatively, the area and the volume of the affected area A are obtained by detecting a position where the luminescence in the ordinary captured image is radically changed as a contour of the affected area A, extracting an area surrounded by the contour as the affected area A, and measuring the area or the volume of the affected area A.

The temperature of the affected area A is obtained according to an infrared image. In this case, an imaging element 9b having sensitivity to an infrared light is used.

In the first and the second embodiments, a marker indicating the affected area A in which the determination section 14 determines that the phototherapy is completed may be superimposed in the endoscope image.

When there are a plurality of the affected areas A in the body, the doctor starts to apply the phototherapy to another affected area A by moving the insertion section 7 so as to move a field of vision of the endoscope 1 after the phototherapy in one affected area A has been completed. By applying the marker to the affected area A in which the phototherapy has been completed, the doctor can easily recognize whether or not the phototherapy in the affected area A has been completed when moving the field of vision of the endoscope 1.

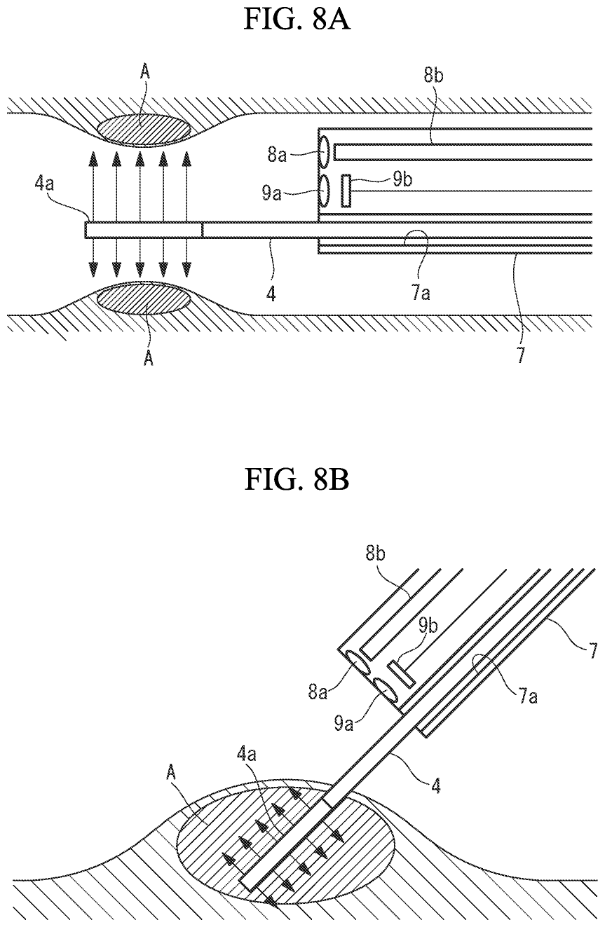

In the first and the second embodiments, the distal end of the probe 4 is arranged at a position opposite to the affected area A, and the therapeutic light L2 and the detection light L3 are irradiated onto the affected area A from the distal end of the probe 4, however, the method for irradiating the therapeutic light L2 and the detection light L3 onto the affected area A is not limited hereto, and it is changeable as needed. FIGS. 8A and 8B show another example of a state of using the phototherapy systems 100, 200.

FIG. 8A explains the phototherapy applied to an affected area A which is formed on an inner wall of a lumen. A distal end portion 4a of the probe 4 protruding from the distal end of the insertion section 7 is placed at a position in the lumen, and the light L2, L3 are radially generated from a side surface of the distal end potion 4a to the surrounding affected area A. Accordingly, a side light emission optical fiber which generates the light from the side surface of the distal end portion 4a is used as the probe 4.

FIG. 8B explains the phototherapy applied to an affected area A formed on a surface of a tissue. The distal end portion 4a of the probe 4 protruding from the distal end of the insertion section 7 is inserted into the affected area A so as to radially generates the light L2 and L3 from a side surface of the distal end portion 4a, which is inserted into the affected area A, to the surrounding affected area A. In this case also, the side light emission optical fiber is used as the probe 4.

According to the present disclosure, it is possible to achieve an effect of assisting determination as to whether to end phototherapy applied to an affected area.

### REFERENCE SIGNS LIST

100, 200 phototherapy system
1 endoscope
2 illumination light source
3 therapeutic light source
4 probe (detection light irradiation section, blood flow related information obtaining section)
5 image processor
6 monitor, indication section
9 optical imaging system (light detection section, image obtaining section)
10 phototherapy assistance device
11 detection light source
12 light detection section (blood flow related information obtaining section)
13, 16 calculation section (blood flow related information obtaining section)
14 determination section (information generating section)
15 light amount adjustment section
17 color detection section
A affected area
L1 illumination light
L2 therapeutic light
L3, L3′ detection light

1. A phototherapy assistance method for assisting phototherapy applied to an affected area using a photo-responsive medical agent, the phototherapy assistance method comprising:
obtaining blood flow related information regarding blood flow of the affected area based on reflected light of detection light that is irradiated onto the affected area when therapeutic light is being irradiated onto the affected area, a wavelength of the detection light being different from that of the therapeutic light irradiated onto the affected area to which the a photo-responsive medical agent is applied;
generating progression degree related information regarding progression degree of the phototherapy in the affected area by associating the blood flow related information with the progression degree; and
indicating the progression degree related information.

2. The phototherapy assistance method according to claim 1, wherein
the blood flow related information is one of a blood flow rate and a number of capillary vessels of the affected area which are used as a parameter for determination, and
the progression degree related information can be obtained by determining whether or not the parameter for determination reaches a predetermined threshold.

3. The phototherapy assistance method according to claim 2, wherein the obtaining of the blood flow related information comprises:
irradiating detection light for detecting the blood flow rate or the number of the capillary vessels onto the affected area;
detecting the detection light reflected by the affected area; and
calculating the blood flow rate or the number of the capillary vessels based on the detected detection light.

4. The phototherapy assistance method according to claim 3, wherein
the detecting the detection light includes detecting an amount of the detection light reflected by the affected area, and
the calculating the blood flow rate or the number of the capillary vessels is to calculate the blood flow rate based on the amount of the detection light.

5. The phototherapy assistance method according to claim 3, wherein
the detecting the detection light includes obtaining image information based on the detection light reflected by the affected area, and
the blood flow related information is extracted from the image information.

6. The phototherapy assistance method according to claim 3, wherein the detection light includes blue light.

7. The phototherapy assistance method according to claim 6, wherein the detection light further includes light having a longer wavelength than the blue light.

8. The phototherapy assistance method according to claim 3, wherein the detection light is a pulsed laser beam having a wavelength reflected by blood, and
calculating the blood flow rate or the number of the capillary vessels is to calculate the blood flow rate based on a shifted amount of a frequency of the detected detection light.

9. A phototherapy assistance system for assisting phototherapy applied to the affected area using the photo-responsible medical agent, the phototherapy assistance device comprising:
a blood flow related information obtaining section which obtains blood flow related information regarding blood flow of the affected area based on reflected light of detection light that is irradiated onto the affected area when therapeutic light is being irradiated onto the affected area, a wavelength of the detection light being different from that of the therapeutic light irradiated onto the affected area to which the a photo-responsive medical agent is applied;
an information generating section which associates the blood flow related information with progression degree of the phototherapy in the affected area to generate progression degree related information regarding the progression degree; and
an indication section which indicates the progression degree related information.

10. The phototherapy assistance system according to claim 9, wherein,
the blood flow related information is one of a blood flow rate and a number of capillary vessels of the affected area which are used as a parameter for determination, and
the information generating section obtains the progression degree related information by determining whether or not the parameter for determination reaches a predetermined threshold.

11. The phototherapy assistance system according to claim 10, wherein the blood flow related information obtaining section comprises:
a detection light irradiation section which irradiates detection light for detecting the blood flow rate or the number of the capillary vessels onto the affected area;
a light detection section which detects the detection light reflected by the affected area; and
a calculation section which calculates the blood flow rate or the number of the capillary vessels based on the detection light detected by the light detection section.

12. The phototherapy assistance system according to claim 11, wherein
the light detection section detects an amount of the detection light reflected by the affected area, and
the calculation section calculates the blood flow rate based on the amount of the detection light detected by the light detection section.

13. The phototherapy assistance system according to claim 10, wherein the blood flow related information obtaining section sets the predetermined threshold based on the blood flow related information at a start of the phototherapy.

14. The phototherapy assistance system according to claim 11, wherein the detection light is a pulsed light or light having a wavelength which is different from a therapeutic light.

15. The phototherapy assistance system according to claim 9, wherein the blood flow related information is a color of the affected area,
wherein the blood flow related information obtaining section comprises:
an image obtaining section which obtains a white light image of the affected area; and
a color detection section which detects the color of the affected area from the white light image obtained by the image obtaining section.

16. The phototherapy assistance system according to claim 10 further comprising:
a light amount adjustment section which adjusts an amount of therapeutic light irradiated onto the affected area,
wherein the light amount adjustment section reduces the amount of the therapeutic light when the information generating section determines that the parameter for determination reaches a predetermined threshold.

17. A processor for assisting phototherapy applied to the affected area using the photo-responsible medical agent, the phototherapy assistance device comprising:
a blood flow related information obtaining section which obtains blood flow related information regarding blood flow of the affected area based on reflected light of detection light that is irradiated onto the affected area when therapeutic light is being irradiated onto the affected area, a wavelength of the detection light being different from that of the therapeutic light irradiated onto the affected area to which the a photo-responsive medical agent is applied;
an information generating section which associates the blood flow related information with progression degree of the phototherapy in the affected area to generate progression degree related information regarding the progression degree; and
an indication section which indicates the progression degree related information generated by the information generating section.

18. The processor according to claim 17, wherein,
the blood flow related information is one of a blood flow rate and a number of capillary vessels of the affected area which are used as a parameter for determination, and
the information generating section obtains the progression degree related information by determining whether or not the parameter for determination reaches a predetermined threshold.

19. The processor according to claim 18, wherein the blood flow related information obtaining section comprises:
a detection light irradiation section which irradiates detection light for detecting the blood flow rate or the number of the capillary vessels onto the affected area;
a light detection section which detects the detection light reflected by the affected area; and
a calculation section which calculates the blood flow rate or the number of the capillary vessels based on the detection light detected by the light detection section.

20. The processor according to claim 19, wherein
the light detection section detects an amount of the detection light reflected by the affected area, and
the calculation section calculates the blood flow rate based on the amount of the detection light detected by the light detection section.
